(19) United States
(12) Patent Application Publication
(10) Pub. No.: US 2024/0141854 A1
(43) Pub. Date: May 2, 2024

(54) AIR CLEANERS AND AIR DISINFECTORS FOR MASS TRANSIT VEHICLES

(71) Applicant: Mobile Climate Control, Corp., York, PA (US)

(72) Inventors: Chang Ching, York, PA (US); Alak Sarker, York, PA (US); Brent Griffith, York, PA (US)

(21) Appl. No.: 17/977,258

(22) Filed: Oct. 31, 2022

(51) Int. Cl. F02M 35/02; A61L 9/20; F02M 35/024

(52) U.S. Cl. CPC F02M 35/0201; A61L 9/20; F02M 35/024

(57) ABSTRACT

An air cleaner is adapted to be mounted inside of a cabin of a mass transit vehicle to remove or disinfect pathogens in the air. The air cleaner includes a housing, an air filter, a grill, and optionally a disinfector. The housing is formed to define an air chamber, an inlet into the air chamber, and an outlet out of the air chamber. A fan draws air through the air chamber during use of the air cleaner. The air filter is received in the air chamber and the grill covers the air filter to secure the air filter between the grill and the housing and allow air to flow through the grill and filter into the air chamber.

### FIELD OF THE DISCLOSURE

The present disclosure relates generally to air cleaners, filters, and disinfectors and, more specifically, to air cleaners, filters, and disinfectors for mass transit vehicles.

### BACKGROUND

Typical mass transit vehicles, such as buses, school buses, shuttles, have a HVAC systems for heating and cooling the cabin of the vehicle where passengers are located. The HVAC systems may have an air filter to remove large particles from the air. However, there remains a desire to remove additional particulate and disinfect pathogens from the air in the cabins to prevent infection and improve health of the passengers and the environmental quality of the air inside of the cabin of the vehicle.

### SUMMARY

The present disclosure may comprise one or more of the following features and combinations thereof.

According to an aspect of the disclosure, an air cleaner is adapted to be mounted inside of a cabin of a mass transit vehicle. The air cleaner includes a housing, an air filter, and a grill. The housing a base, a cover, and a filter holder. The base defines an air chamber arranged to receive air to be cleaned by the air cleaner. The cover is coupled with the base to close off access to the air chamber and is formed to define an inlet for the air to move into the air chamber and an outlet for the air to move out of the air chamber. The filter holder is located within the air chamber and coupled with the cover to align the filter holder with the inlet of the cover such that the air moving into the air chamber passes through the filter holder during use of the air cleaner.

The air filter is engaged with the filter holder such that the air filter is maintained in a path of the air moving through the inlet during use of the air cleaner so that the air is conducted through the air filter. The grill is configured to regulate a flow of the air that moves through the inlet and the air filter into the air chamber during use of the air cleaner. The grill is removably coupled with the cover to trap the air filter between the grill and the filter holder and block the air filter from being removed from the air chamber.

In some embodiments, the cover includes a cover panel coupled with the base and a fan coupled with the cover panel. The fan is configured to draw the air from outside of the housing through the grill, the inlet, and the air filter into the air chamber and then out of the air chamber through the outlet to remove particles from the air in the cabin of the mass transit vehicle.

In some embodiments, the grill is formed to define a plurality of apertures that extend through the grill. Each of the plurality of apertures have a same cross-sectional area. The plurality of apertures are spaced apart from one another with a varying spacing distance to regulate the flow of the air moving into the air chamber.

In some embodiments, the plurality of apertures includes a first aperture located fluidly nearest the fan, a second aperture that directly neighbors the first aperture, a third aperture located fluidly furthest from the fan, and a fourth aperture that directly neighbors the third aperture. The varying spacing distance is greatest in magnitude between the first aperture and the second aperture and smallest in magnitude between the third aperture and the fourth aperture.

In some embodiments, the plurality of apertures includes a first set of apertures spaced apart from one another by a first spacing distance, a second set of apertures spaced apart from one another by a second spacing distance, and a third set of apertures spaced apart from one another by a third spacing distance. The second set of apertures are located between the first set of apertures and the third set of apertures. The first spacing distance is greater than the second spacing distance. The second spacing distance is greater than the third spacing distance.

In some embodiments, the base includes a bottom wall, a first side wall, a second side wall, and a partition wall. The first side wall extends away from the bottom wall. The second side wall extends away from the bottom wall and is spaced apart from the first side wall. The partition wall contacts and extends away from the first side wall toward the second side wall and terminates at an end that is spaced apart from the second side wall to partition the air chamber.

In some embodiments, the inlet is located on a first side of the partition wall. The outlet is located on a second side of the partition wall opposite the first side of the partition wall so that the air moving through the air chamber is directed through the inlet and along the first side of the partition wall, around the end of the partition wall, and then along the second side of the partition wall before being directed out of the air chamber through the outlet.

In some embodiments, the fan is directly aligned with the outlet. In some embodiments, the filter holder is formed to define a U-shaped channel and is formed to include a plurality of openings that extend through the filter holder to allow the air to pass through the filter holder. In some embodiments, a length of the inlet is approximately 80 percent or more of the length of the air chamber.

In some embodiments, the air cleaner includes an air disinfector located within the air chamber. The air disinfector is configured to disinfect the air moving through the air chamber during use of the air cleaner. The air disinfector includes an ultraviolet light source configured to irradiate the air moving through the air chamber during use of the air cleaner.

According to another aspect of the present disclosure, an air cleaner includes a housing, an air filter, and grill. The housing includes a base, a cover panel, a fan, and a filter holder. The base is formed to define an air chamber therein. The cover panel is arranged to close off access to the air chamber and formed to define an inlet into the air chamber and an outlet out of the air chamber. The fan is configured to draw air through the air chamber. The filter holder is aligned with the inlet of the cover panel. The air filter is engaged with the filter holder. The grill is coupled with the cover panel to trap the air filter between the grill and the filter holder.

In some embodiments, the air cleaner further includes an air disinfector located within the air chamber. The air disinfector is configured to disinfect the air moving through the air chamber during use of the air cleaner.

In some embodiments, a length of the inlet is approximately 80 percent or more of the length of the air chamber. In some embodiments, the grill is formed to define a plurality of apertures. Each of the plurality of apertures have a same cross-sectional area. The plurality of apertures being spaced apart from one another from a first end of the grill to a second end of the grill with a varying spacing distance between the plurality of apertures.

In some embodiments, the plurality of apertures includes a first set of apertures spaced apart from one another by a first spacing distance, a second set of apertures spaced apart from one another by a second spacing distance, and a third set of apertures spaced apart from one another by a third spacing distance. The second set of apertures are located between the first set of apertures and the third set of apertures. The first spacing distance is greater than the second spacing distance. The second spacing distance is greater than the third spacing distance.

In some embodiments, the base includes a bottom wall, a first side wall, a second side wall, and a partition wall. The first side wall extends away from the bottom wall. The second side wall extends away from the bottom wall and is spaced apart from the first side wall. The partition wall contacts and extends away from the first side wall toward the second side wall and terminates at an end that is spaced apart from the second side wall to partition the air chamber. The inlet is located on a first side of the partition wall and the outlet is located on a second side of the partition wall opposite the first side of the partition wall.

In some embodiments, the filter holder is formed to define a U-shaped channel. The filter holder is formed to include a plurality of openings that extend through the filter holder to allow the air to pass through the filter holder.

According to another aspect of the present disclosure, a method of operating an air cleaner includes a number of steps. The method includes providing a housing having a base that defines an air chamber and that includes a bottom wall, a first side wall that extends away from the bottom wall, a second side wall that extends away from the bottom wall and spaced apart from the first side wall, and a partition wall that contacts and extends away from the first side of the partition wall toward the second side wall and terminates at an end that is spaced apart from the second side wall to partition the air chamber, providing a cover panel arranged to close off access to the air chamber, an air filter engaged received in the air chamber, and a grill coupled with the cover panel to trap the air filter between the grill and the housing, drawing air through an inlet of the grill and filter into the air chamber on a first side of the partition wall, directing the air along the first side of the partition wall, around the end of the partition wall, and along a second side of the partition wall, and moving the air from the second side of the partition wall through an outlet of the grill and out of the air cleaner.

In some embodiments, the grill is formed to define a plurality of apertures. Each of the plurality of apertures have a same cross-sectional area. The plurality of apertures are spaced apart from one another from a first end of the grill to a second end of the grill with a varying spacing distance between the plurality of apertures. In some embodiments, the method further includes disinfecting the air as the air is directed along the second side of the partition wall.

These and other features of the present disclosure will become more apparent from the following description of the illustrative embodiments.

### BRIEF DESCRIPTION OF THE DRAWINGS

FIG. 1 is an elevation view of a mass transit vehicle, the vehicle including a frame and a cabin adapted to transport passengers within the mass transit vehicle, an air cleaner in accordance with the present disclosure is shown mounted to a wall inside of the cabin;

FIG. 2 is a perspective view of the inside of the cabin of the vehicle of FIG. 1 showing that the cabin includes a plurality of seats and standing room for the passengers as well as the air cleaner mounted to the wall of the cabin for cleaning the air circulating within the cabin;

FIG. 3 is a perspective view of the air cleaner mounted in the mass transit vehicle of FIGS. 1 and 2, the air cleaner including a housing that defines an air chamber therein, an air filter located within the air cleaner, and a grill that covers the air filter and allows air to flow through the grill and air filter into and through the housing to clean the air including the removal or disinfection of pathogens from the air;

FIG. 4 is an exploded view of the air cleaner of FIG. 3 showing the air cleaner includes, from top to bottom, the grill, a cover panel of the housing, the air filter, and an air filter holder, a fan, and a base of the housing;

FIG. 5 is a section view of the air cleaner of FIG. 3 showing the base is partitioned to draw air through the grill and the air filter into a first side of the air chamber and move the air out of the base through a fan on a second side of the air chamber;

FIG. 6 is a perspective view of the air cleaner of FIG. 3 with the grill and air filter removed to reveal the filter holder of the housing and to show the filter holder is formed to include a plurality of large apertures so that the filter holder is able to support the air filter in the filter holder while allowing the air to pass through the filter holder and enter into the air chamber formed in the housing;

FIG. 7 is a top elevation view of the air cleaner of FIG. 3 showing the grill includes a plurality of apertures, each of the apertures being of the same shape and cross-sectional area, and a spacing between the apertures varies from left to right to control the air flow passing through the grill and into the air chamber; and

FIG. 8 is a bottom elevation view of the filter for the air cleaner showing that a gasket layer is arranged along the perimeter of the filter and the gasket layer is configured to block air from passing around the air filter.

### DETAILED DESCRIPTION OF THE DRAWINGS

For the purposes of promoting an understanding of the principles of the disclosure, reference will now be made to a number of illustrative embodiments illustrated in the drawings and specific language will be used to describe the same.

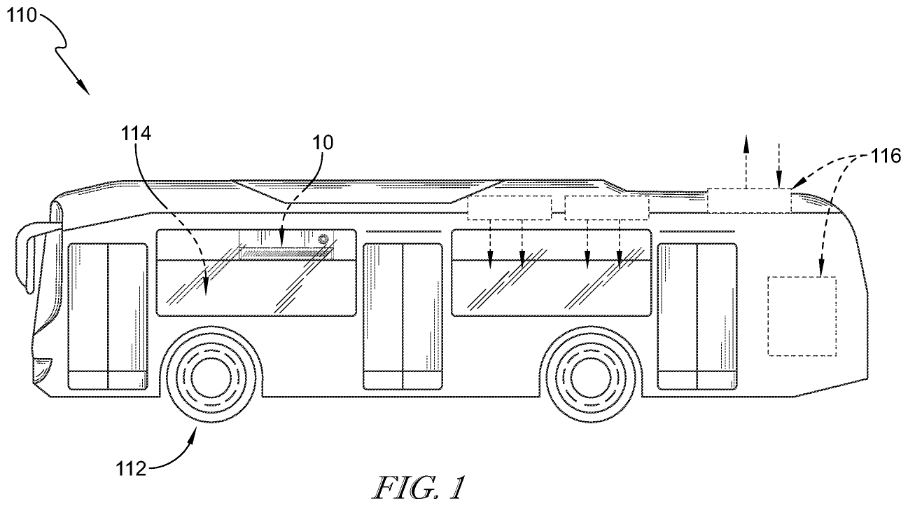

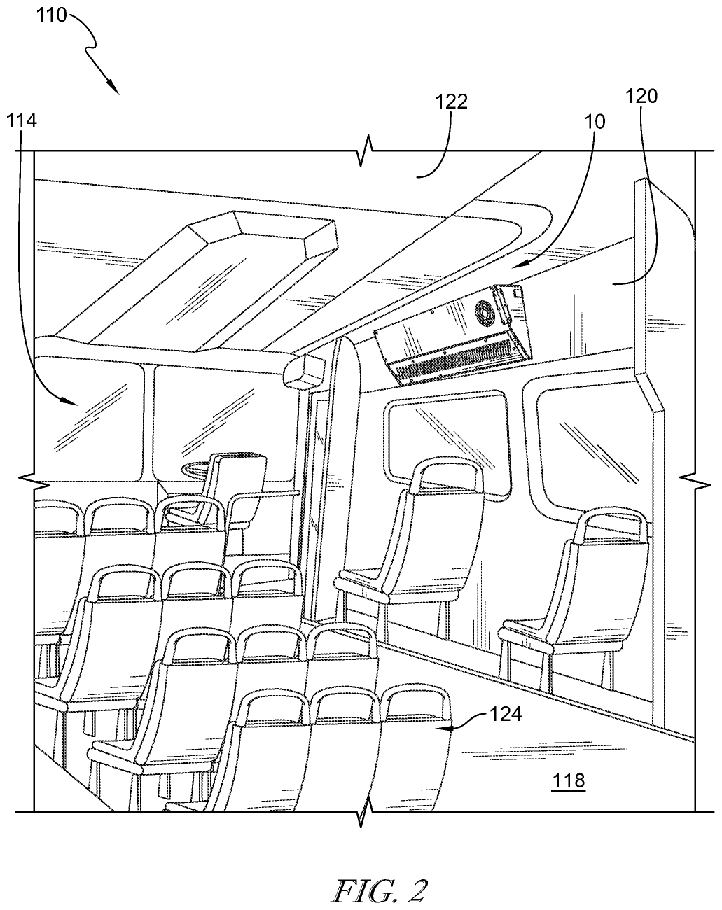

An air cleaner 10 is adapted for use with a mass transit vehicle 110 to clean the air within a passenger cabin 114 of the vehicle 110 as shown in FIGS. 1 and 2. The air cleaner 10 is configured to remove pathogens by filtering droplets, aerosols, etc. laden with pathogens. The mass transit vehicle 110 may be, for example, a bus, coach, train, school bus, shuttle, etc. The air cleaner 10 is mountable within the cabin 114, for example, on a wall or ceiling of the vehicle 110. In the illustrative embodiment, the air cleaner 10 is self-contained such that it is independently operable from other systems of the vehicle 110 so long as electrical power is delivered to the air cleaner 10. The air cleaner 10 may be mounted during manufacture of the vehicle 110 or post-production on an existing vehicle 110.

Typical mass transit vehicles have HVAC systems, such as system 116 in FIG. 1, for heating and cooling the cabin of the vehicle 110. The HVAC systems may have an air filter to remove large particles from the air. However, there remains a desire to remove additional particles and/or disinfect the air in the cabin beyond the capabilities of a typical HVAC system 116.

The air cleaner 10 is coupled to the vehicle 110 within the cabin 114 of the vehicle 110, during manufacture or after-market, and cleans and/or disinfects the air within the cabin 114 as suggested in FIG. 2. The air cleaner 10 removes or disinfects particles wherein particles are understood to include droplets, aerosols, pathogens, dirt, pollen, mold, etc. In the illustrative embodiment, the vehicle 110 is a bus 110 and the electronic components of the air cleaner 10 are powered by an electric energy source included in the bus 110. In some embodiments, the vehicle 110 is powered by all-electric motor, an internal combustion engine, or a hybrid internal combustion engine and electric power source. As shown in FIG. 1, the vehicle 110 includes a frame 112 with a body, engine/motor, drive train, wheels, tires, etc., the cabin 114 coupled with the frame, and the HVAC system 116 configured to heat and cool air that is conducted into the cabin 114.

The cabin 114 includes a floor 118, walls 120, a ceiling 122, and a plurality of seats 124 coupled with the floor 118 as shown in FIG. 2. The air cleaner 10 is mounted on one of the walls 120 in the cabin 114. The air cleaner 10 is mounted high on the wall at or above the heads of the passengers standing in the cabin 114. In other embodiments, the air cleaner is mounted lower on the wall 120 or on the ceiling 122.

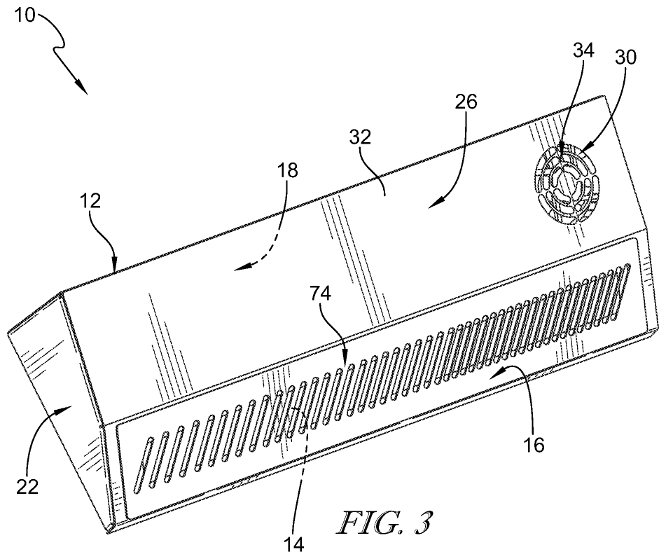

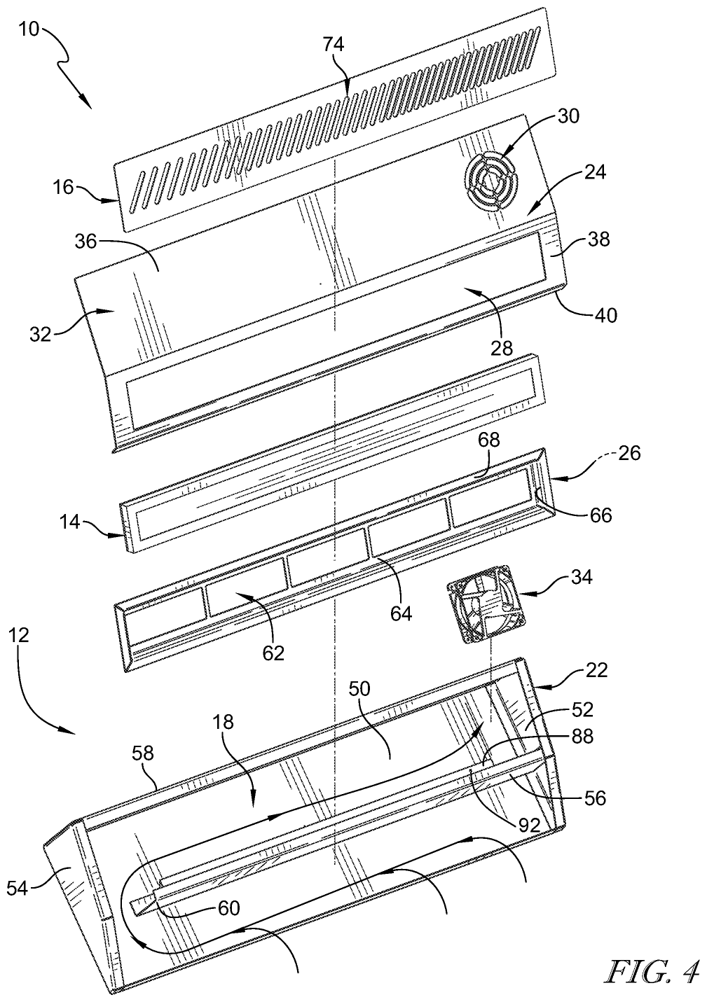

The air cleaner 10 includes a housing 12, an air filter 14, and a grill 16 as shown in FIGS. 3 and 4. The housing 12 is arranged to define an air chamber 18 formed therein to receive air from the cabin 114 to be cleaned and returned to the cabin 114. The air filter 14 is received in the air chamber 18 and is configured to filter out particulates from the air and clean the air passing through the housing 12. The grill 16 is coupled to the housing 12 and covers the air filter 14 while allowing the air to flow through the grill 16 and into the air chamber 18.

The housing includes a base 22, a cover 24, and a filter holder 26 as shown in FIGS. 4-6. The base 22 defines the air chamber 18 arranged to receive the air to be cleaned by the air cleaner 10 as suggested by the arrows in FIG. 4. The cover 24 is coupled with the base 22 to close off access to the air chamber 18. The cover 24 is formed to define an inlet 28 for the air to move into the air chamber 18 and an outlet 30 for the air to move out of the air chamber 18. The filter holder 26 is located within the air chamber 18 and coupled with the cover 24 to align the filter holder 26 with the inlet 28 of the cover 24 such that the air moving into the air chamber 18 passes through the filter holder 26 during use of the air cleaner 10.

The base includes a bottom wall 50, a first side wall 52, a second side wall 54, a partition wall 56, and a back wall 58 as shown in FIG. 4. The first side wall 52 extends away from the bottom wall 50. The second side wall 54 extends away from the bottom wall 50 and is spaced apart from the first side wall 52 at an opposite end of the bottom wall 50. The partition wall 56 contacts and extends away from the first side wall 52 toward the second side wall 54 and terminates at an end 60 that is spaced apart from the second side wall 54 to partition the air chamber 18.

The inlet 28 into the air chamber 18 is located on a first side of the partition wall 56 as suggested in FIGS. 4-6. The outlet 30 is located on a second side of the partition wall 56 opposite the first side of the partition wall 56 so that the air moving through the air chamber 18 is directed through the inlet 28 and along the first side of the partition wall 56, around the end 60 of the partition wall 56, and then along the second side of the partition wall 56 before being directed out of the air chamber 18 through the outlet 30 as suggested by the arrows in FIG. 4. The outlet 30 is located adjacent the first side wall 52 and away from the end 60 of the partition wall 56.

The cover 24 includes a cover panel 32 and a fan 34 or other air moving device such as a blower, etc. as shown in FIG. 4. The cover panel 32 is a paneling coupled with the base 22 to block off the air chamber 18. The cover panel 32 includes a planar first panel 36, a planar second panel 38 at an angle relative to the first panel 36, and a planar third panel 40 that extends from the second panel 38 at an angle. The fan 34 is coupled with the cover panel 32 and configured to draw the air from outside of the housing 12 through the grill 16, the inlet 28, and the air filter 14 into the air chamber 18 and then out of the air chamber 18 through the outlet 30 to remove particles from the air in the cabin 114 of the mass transit vehicle 110. The fan 34 is directly aligned with the outlet 30. The fan 34 may include a lock out feature such as a locking-rocker switch.

The cover panel 32 is formed to define the inlet 28 and the outlet 30. The inlet 28 is formed in the second panel 38 and is rectangular in shape. In the illustrative embodiment, the inlet 28 has a length 46 that is about 80 percent or more of a length 48 of the cover panel 32 and the base 22 as suggested in FIG. 6. In some embodiments, the length 46 is about 50 to about 95 percent of the length 48. In some embodiments, the length 46 is about 75 to about 95 percent of the length 48. In some embodiments, the length 46 is about 75 to about 90 percent of the length 48. In some embodiments, the length 46 is at least about 50 percent of the length 48. In some embodiments, the length of the inlet is approximately 90 percent or more of the length of the air chamber. In some embodiments, the length of the inlet is approximately 95 percent or more of the length of the air chamber.

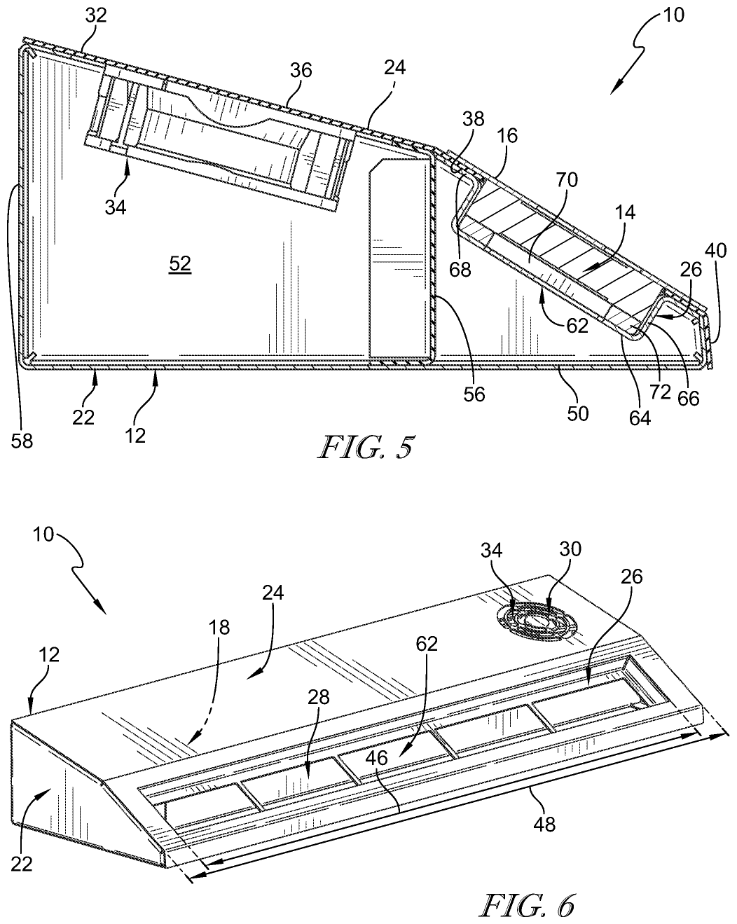

The filter holder 26 is formed to define a U-shaped channel and is formed to include a plurality of openings 62 that extend through the filter holder 26 to allow the air to pass through the filter holder 26 as shown in FIGS. 4 and 6. The filter holder 26 includes an inner wall 64 formed to define the openings 62, a side wall 66, and a perimeter wall 68. The side wall 66 extends away from the inner wall 64 and interconnects the inner wall 64 and the perimeter wall 68. The perimeter wall 68 extends away from the side wall 66 and provides a surface that mounts with the cover panel 32 as shown in FIG. 5.

The air filter 14 is engaged with and received in the filter holder 26 as shown in FIG. 5. The air filter 14 is maintained in a path of the air moving through the inlet 28 during use of the air cleaner 10 so that the air is conducted through the air filter 14 and particles are removed from the air by the air filter 14. The air filter 14 includes a filter element 70 and a gasket 72 as shown in FIG. 8. The filter element 70 may include a fibrous and/or porous material configured to trap particles as the air passes through the filter element 70. The gasket 72 is arranged along a perimeter of the filter element 70 and engages the filter element 70 and the inner wall 64 of the filter holder 26. In some embodiments, the gasket 72 is coupled with the inner wall 64 and in some embodiments, the gasket 72 is coupled with the filter element 70. The gasket 72 blocks air from passing around the filter element 70 and the filter holder 26 without passing through the filter element 70.

The grill 16 is configured to regulate the flow of the air that moves through the inlet 28 and the air filter 14 into the air chamber 18 during use of the air cleaner 10 as suggested in FIGS. 3-5. The grill 16 is removably coupled with the cover 24 to trap the air filter 14 between the grill 16 and the filter holder 26 and block the air filter 14 from being removed from the air chamber 18. The grill 16 is configured to be separated from the cover 24 to allow access to and replacement of the air filter 14 as well as access to the filter holder 26 and air chamber 18.

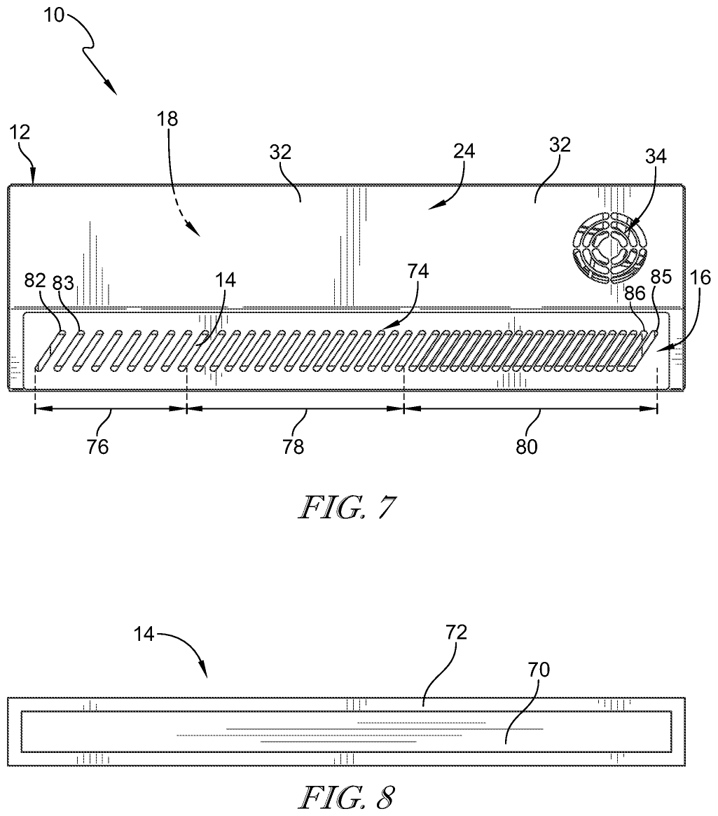

The grill 16 is a planar rectangular panel in the illustrative embodiment. The grill 16 is formed to define a plurality of apertures 74 that extend through the grill 16. Each of the plurality of apertures 74 have a same cross-sectional area in the illustrative embodiment in which they are elongated slots. The plurality of apertures 74 are spaced apart from one another with a varying spacing distance to regulate the flow of the air moving into the air chamber 18 as suggested in FIG. 7.

The plurality of apertures 74 includes a first set of apertures 76 spaced apart from one another by a first spacing distance, a second set of apertures 78 spaced apart from one another by a second spacing distance, and a third set of apertures 80 spaced apart from one another by a third spacing distance as shown in FIG. 7. The second set of apertures 78 is located between the first set of apertures 76 and the third set of apertures 80. The first spacing distance is greater than the second spacing distance and the second spacing distance is greater than the third spacing distance.

The different spacing distances provides a desired flow rate into the air chamber 18 along the length 46 of the inlet 28. The fan 34 provides a greater draw pressure fluidly nearest the fan 34 and relatively lower draw pressure fluidly furthest from the fan 34. As such, there first spacing distance for the first set of apertures 76 is greater than the second and third spacing distances so that a more uniform flow of air enters through all of the sets of apertures 76, 78, 80.

In some embodiments, the spacing between the apertures 74 varies continuously from one side of the inlet 28 to the other side of the inlet 30. In some embodiments, the plurality of apertures 74 includes a first aperture 82 located fluidly nearest the fan 34, a second aperture 83 that directly neighbors the first aperture 82, a third aperture 85 located fluidly furthest from the fan 34, and a fourth aperture 86 that directly neighbors the third aperture 86. The varying spacing distance is greatest in magnitude between the first aperture 82 and the second aperture 83 and smallest in magnitude between the third aperture 85 and the fourth aperture 86.

The air cleaner 10 further includes an air disinfector 88 in the illustrative embodiment as shown in FIG. 4. The air disinfector 88 is located within the air chamber 18 and configured to disinfect the air moving through the air chamber 18 during use of the air cleaner 10. The air disinfector 88 includes an ultraviolet light source configured to irradiate the air moving through the air chamber 18 during use of the air cleaner 10. In the illustrative embodiment, the air disinfector 88 is coupled with the partition wall 56 on the second side of the partition wall 56. As a result, the light or other disinfecting source of the air disinfector 88 is blocked from illuminating or be exposed to the passengers. In some embodiments, the air disinfector 88 includes an ion generator, a hydrogen peroxide generator, or a chlorine dioxide dispenser. In the illustrative embodiment, a switch 92 or other implementation is connected with the air disinfector 88 to automatically disable the air disinfector 88 in response to one of the cover 24 being separated from the base 22.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

What is claimed is:

1. An air cleaner adapted to be mounted inside of a cabin of a mass transit vehicle, the air cleaner comprising
a housing that includes a base, a cover, and a filter holder, the base defines an air chamber arranged to receive air to be cleaned by the air cleaner, the cover being coupled with the base to close off access to the air chamber and formed to define an inlet for the air to move into the air chamber and an outlet for the air to move out of the air chamber, and the filter holder being located within the air chamber and coupled with the cover to align the filter holder with the inlet of the cover such that the air moving into the air chamber passes through the filter holder during use of the air cleaner,
an air filter engaged with the filter holder such that the air filter is maintained in a path of the air moving through the inlet during use of the air cleaner so that the air is conducted through the air filter, and
a grill configured to regulate a flow of the air that moves through the inlet and the air filter into the air chamber during use of the air cleaner, the grill being removably coupled with the cover to trap the air filter between the grill and the filter holder and block the air filter from being removed from the air chamber,
wherein the cover includes a cover panel coupled with the base and a fan coupled with the cover panel, the fan being configured to draw the air from outside of the housing through the grill, the inlet, and the air filter into the air chamber and then out of the air chamber through the outlet to remove particles from the air in the cabin of the mass transit vehicle.

2. The air cleaner of claim 1, wherein the grill is formed to define a plurality of apertures that extend through the grill, each of the plurality of apertures having a same cross-sectional area, and the plurality of apertures being spaced apart from one another with a varying spacing distance to regulate the flow of the air moving into the air chamber.

3. The air cleaner of claim 2, wherein the plurality of apertures includes a first aperture located fluidly nearest the fan, a second aperture that directly neighbors the first aperture, a third aperture located fluidly furthest from the fan, and a fourth aperture that directly neighbors the third aperture and wherein the varying spacing distance is greatest in magnitude between the first aperture and the second aperture and smallest in magnitude between the third aperture and the fourth aperture.

4. The air cleaner of claim 2, wherein the plurality of apertures includes a first set of apertures spaced apart from one another by a first spacing distance, a second set of apertures spaced apart from one another by a second spacing distance, and a third set of apertures spaced apart from one another by a third spacing distance, the second set of apertures located between the first set of apertures and the third set of apertures, the first spacing distance being greater than the second spacing distance, and the second spacing distance being greater than the third spacing distance.

5. The air cleaner of claim 4, wherein the base includes a bottom wall, a first side wall that extends away from the bottom wall, a second side wall that extends away from the bottom wall and spaced apart from the first side wall, and a partition wall that contacts and extends away from the first side wall toward the second side wall and terminates at an end that is spaced apart from the second side wall to partition the air chamber.

6. The air cleaner of claim 5, wherein the inlet is located on a first side of the partition wall and the outlet is located on a second side of the partition wall opposite the first side of the partition wall so that the air moving through the air chamber is directed through the inlet and along the first side of the partition wall, around the end of the partition wall, and then along the second side of the partition wall before being directed out of the air chamber through the outlet.

7. The air cleaner of claim 6, wherein the fan is directly aligned with the outlet.

8. The air cleaner of claim 7, wherein the filter holder is formed to define a U-shaped channel and is formed to include a plurality of openings that extend through the filter holder to allow the air to pass through the filter holder.

9. The air cleaner of claim 8, wherein a length of the inlet is approximately 80 percent or more of the length of the air chamber.

10. The air cleaner of claim 1, further comprising an air disinfector located within the air chamber and configured to disinfect the air moving through the air chamber during use of the air cleaner and the air disinfector includes an ultraviolet light source configured to irradiate the air moving through the air chamber during use of the air cleaner.

11. An air cleaner adapted to be mounted inside of a cabin of a mass transit vehicle, the air cleaner comprising
a housing that includes a base formed to define an air chamber, a cover panel arranged to close off access to the air chamber and formed to define an inlet into the air chamber and an outlet out of the air chamber, a fan configured to draw air through the air chamber, and a filter holder aligned with the inlet of the cover panel,
an air filter engaged with the filter holder, and
a grill coupled with the cover panel to trap the air filter between the grill and the filter holder.

12. The air cleaner of claim 11, further comprising an air disinfector located within the air chamber and configured to disinfect the air moving through the air chamber during use of the air cleaner.

13. The air cleaner of claim 11, wherein a length of the inlet is approximately 80 percent or more of the length of the air chamber.

14. The air cleaner of claim 13, wherein the grill is formed to define a plurality of apertures, each of the plurality of apertures having a same cross-sectional area, and the plurality of apertures being spaced apart from one another from a first end of the grill to a second end of the grill with a varying spacing distance between the plurality of apertures.

15. The air cleaner of claim 14, wherein the plurality of apertures includes a first set of apertures spaced apart from one another by a first spacing distance, a second set of apertures spaced apart from one another by a second spacing distance, and a third set of apertures spaced apart from one another by a third spacing distance, the second set of apertures are located between the first set of apertures and the third set of apertures, the first spacing distance is greater than the second spacing distance, and the second spacing distance is greater than the third spacing distance.

16. The air cleaner of claim 11, wherein the base includes a bottom wall, a first side wall that extends away from the bottom wall, a second side wall that extends away from the bottom wall and spaced apart from the first side wall, and a partition wall that contacts and extends away from the first side wall toward the second side wall and terminates at an end that is spaced apart from the second side wall to partition the air chamber, and
wherein the inlet is located on a first side of the partition wall and the outlet is located on a second side of the partition wall opposite the first side of the partition wall.

17. The air cleaner of claim 11, wherein the filter holder is formed to define a U-shaped channel and is formed to include a plurality of openings that extend through the filter holder to allow the air to pass through the filter holder.

18. A method of operating an air cleaner, the method comprising
providing a housing having a base that defines an air chamber and that includes a bottom wall, a first side wall that extends away from the bottom wall, a second side wall that extends away from the bottom wall and spaced apart from the first side wall, and a partition wall that contacts and extends away from the first side of the partition wall toward the second side wall and terminates at an end that is spaced apart from the second side wall to partition the air chamber,
providing a cover panel arranged to close off access to the air chamber, an air filter engaged received in the air chamber, and a grill coupled with the cover panel to trap the air filter between the grill and the housing,
drawing air through an inlet of the grill and filter into the air chamber on a first side of the partition wall,
directing the air along the first side of the partition wall, around the end of the partition wall, and along a second side of the partition wall, and
moving the air from the second side of the partition wall through an outlet of the grill and out of the air cleaner.

19. The method of claim 18, wherein the grill is formed to define a plurality of apertures, each of the plurality of apertures having a same cross-sectional area, and the plurality of apertures being spaced apart from one another from a first end of the grill to a second end of the grill with a varying spacing distance between the plurality of apertures.

20. The method of claim 18, further comprising disinfecting the air as the air is directed along the second side of the partition wall.
